(19) United States
(12) Patent Application Publication
(10) Pub. No.: US 2019/0059965 A1
(43) Pub. Date: Feb. 28, 2019

(54) Implements for Stabilising Bone Fractures

(71) Applicants: Thomas Gausepohl, Giessen (DE); Özlem Sohrabi, Giessen (DE)

(72) Inventors: Thomas Gausepohl, Giessen (DE); Özlem Sohrabi, Giessen (DE)

(21) Appl. No.: 16/074,837

(22) PCT Filed: Feb. 2, 2017
(86) PCT No.: PCT/DE2017/100071

§ 371 Date: Aug. 2, 2018

(30) Foreign Application Priority Data
Feb. 2, 2016 (DE) 10 2016 101 817.1
Jan. 31, 2017 (DE) 10 2017 101 882.4

(51) Int. Cl. A61B 17/80; A61B 17/82

(52) U.S. Cl. CPC A61B 17/8085; A61B 17/8057; A61B 17/82

(57) ABSTRACT

In the event of bone fractures, in particular comminuted fractures, having multiple bone fracture pieces, a quick and safe operation can be possible when implements (1) are used for stabilizing the affected bone (6). The main body (2) of these implements is an angle profile (15), wherein long recesses (17-19) are provided in the plate side pieces (10, 11). This facilitates any bending that may become necessary about the longitudinal axis (9) and the transverse axis (16). In this way, it is possible to achieve a high level of stability for the operated region, in order to guarantee an early functional exercising of the neighboring joints without risking the bone fragments shifting again.

The invention relates to an implement for surgical treatment, and therein in particular for the stabilization of bone fractures, consisting of a plate-shaped main body in which drilled holes are provided for affixing the main body to the bone by means of screws and which is manufactured from a material suitable for humans.

In principle it is known how to stabilize bone fractures at all regions of the particular bone by the use of plates and other implements. The implements for stabilizing of fractured bones are usually, but not exclusively, installed after the region of the fractured bone has been exposed. Then the procedure is to re-establish the anatomy as closely as possible, that is, to bring the individual bone fragments back to their original location. In this regard it is important to achieve the greatest possible stability through the use of such implements, so as to ensure an early functional exercise of the neighboring joints without risking the bone fragments shifting again. The known implements generally consist of flat metal with drilled holes to accommodate screws which are used to affix the implements to the particular bone. For example, DE 10 2006 142 277 B4 describes an elongated main body for affixing of the bone in a bone fracture, which has an elongated shape and on one end comprises a kind of finger which is intended to surround the bone as tightly as possible. The solution from DE 10 2008 002 389 B4 is similar, in which the elongated middle section is omitted, and where central drilled holes are provided for affixing of the main body to the bone. The implements known from these publications are indeed fundamentally intended for long bones, but in particular for long bones after fractures or osteotomies of rib bones. The thinking behind the positioning of these implements for stabilization of bone fractures is firstly intended to take into account the anatomy of the surrounding soft tissue, inasmuch as access paths which are anatomically meaningful, often only allow a limited exposure of the broken bone fragments. And secondly, biomechanical necessities must also be taken sufficiently into account. The area moment of inertia plays an important role here. The particular flexural loading on the bone should be taken into account by the positioning of the implement. Using the example of the upper arm bone (humerus), the problem of the area moment of inertia becomes particularly clear. With respect to the biomechanical requirement to neutralize the forces acting on the bone, there exist different theories with respect to the most favorable positioning of these plate-shaped implements. On the elbow, for example, a plate positioning is recommended in which two loose plates are affixed at a right angle to each other in order to neutralize the forces in two directions. More recently there also has been a discussion about affixing the plates in parallel to each other at both sides of the bone, which is called a 180° configuration. In this case it turns out that an optimum stabilization of this bone region is not possible without additional measures. The effort involved with several of these plate-shaped implements is considerable. And it must be taken into account that in the case of comminuted fractures, which occur due to accidents such as traffic accidents, work-related accidents or sports accidents, the broken bone fragments can sometimes be displaced considerably. These fragments must be repositioned surgically as closely as possible to their original position, because otherwise significant pain and restricted movement will result. After the broken bone fragments have been surgically returned to their original position, they must be fixed in place there, which is done, as described, by means of plate screws or wires. Even the smallest inaccuracies due to the plate-shaped implements and screws, which do not provide the necessary stabilization and fixation, often lead to considerable pain. On the elbow in particular, the powerful pull of the tricep muscle and the effect of the flexor muscles extending across a joint cause a noticeable problem since they can easily shift the broken bone fragments against each other. Likewise the implements known from WO 2004/045389 A2 and from US 2006/0089648 A1 are not suitable for alleviating the described problems. The solutions known from these two publications disclose the use of two-part plates which can pivot with respect to each other. The disclosures known from WO 02/076317A1, DE 198 02 229A1 and DE 10 2013 104 887 A1 are to be assessed similarly, in which implements and/or plates are proposed which are arranged so as to pivot either in the longitudinal axis or transverse thereto, and thus represent a complicated and difficult to implement solution.

Therefore the invention is based on the problem of creating an implement to be employed for bone fractures which allows a safe, early functional exercise of the adjoining joints, with no danger of renewed shifting of the bone fragments.

The problem is solved according to the invention in that the length and width of the main body is designed to correspond to the bone region being reinforced therewith and to have a resulting angular profile, wherein the two plate legs thereof are shaped to encompass an angle adapted to the associated bone region. With a design of the implement of this kind it is possible to fully satisfy the different biomechanical requirements. Due to the configuration of the angled profile, the main body of the implement attains a favorable stability which makes it possible to affix the broken bone fragments precisely in the position into which they have been surgically placed. It is also a particular advantage that due to these angled profiles, a fast treatment of the occurring comminuted fracture is possible, in particular when these angled profiles are first shaped into a form which fully takes into account the special anatomical circumstances of the particular bone and/or bone region. This is possible even though the angled shape of the main body already has the desired high stability, especially when the corresponding deformations are implemented in advance. Of course, it is also possible to implement any potentially necessary deformations which become known only during the operation at that time. It is also an advantage that the angled profiles rest against the bone only in a line-shape, or in points, respectively, that is, they only slightly impact the blood supply to the bone.

According to one practical embodiment, the invention provides that the main body features elongated recesses arranged at a distance to each other, which are designed to extend from the back formed by the two plate legs up to the edges of the plate legs and to leave a protective separation. With the aid of this kind of design of the main body or of the angled profile, it is possible to undertake flexures in the longitudinal axis simultaneously but also transversely thereto, and more easily than with the solid plate-shaped angled profile, when this proves to be necessary or expedient owing to the anatomy. This design of the main body additionally proves to be an advantage when such adaptations have to be undertaken, or prove to be expedient, during the actual surgery. Whereas the recesses prove to be expedient in the case of any necessary deformations or edge bends, the stability of the overall main body is only thus slightly less, if at all, than for the one-piece design of the main body. Finally, it is an additional advantage that the weight of the implement is reduced overall, which is especially favorable when the implement is to remain on the bone. In addition, the contact surface upon the bone is also favorably reduced.

According to an additional practical embodiment, the invention provides that the recesses feature a width and spacing to each other that facilitates the edging or bending of the main body about the longitudinal axis and/or a deformation thereof transverse to this axis. Thus according to the invention it is clear that we are dealing not only with tiny notches, but rather with clearly visible and effective “recesses.”

Another embodiment of the implement which further facilitates the treatment of the bone is that the plate legs feature unevennesses distributed along the plate side resting against the bone. These unevennesses mean that there is only point by point contact of the angled profile, wherein due to the specific arrangement of the unevennesses, the particular formation of the bone can be specifically accommodated.

It is an additional advantage that the unevennesses are designed as nipple-like knobs or also as conical or pyramid-like or cube-like protrusions or even as grooves. These embodiments mean that the formation of the entire angled profile can be undertaken such that an implement precisely adapted to the particular circumstances can be created, which as mentioned, further ensures an optimum treatment of the bone.

For reasons of stability and installation it is an advantage that the angled profile according to the invention is designed with an included angle of 90°. Thus in the usual case an optimum positioning of the angled profile against the bone being stabilized is possible.

According to an additional practical embodiment, the invention provides that the angled profile features an angle greater than or less than 90°, adapted to the anatomical install site, wherein the angle can also vary at individual locations of the angled profile. As mentioned above, since the angled profile can be deformed with suitable tools according to the particular install site, the setting of the angle during the installation is possible. But in particular it is possible to adjust the required angle of the corresponding angled profiles in advance, in accordance with the particular install site. Then finally, it would even be possible to design the main body and/or the angled profile to the fracture of the particular patient, or to the averaged anatomical data of several patients, in order thus to provide an optimum design of the angled profile for the user.

The already discussed deformation of the angled profile according to the particular install sites is facilitated in that the recesses are distributed regularly along the back of the angled profile or are concentrated at defined sites and are not provided at all at other sites. This is particularly advantageous if a quite specific adaptation should become necessary during the installation.

With the particular distribution of the recesses across the length of the angled profile, both a change in the longitudinal axis and also in the transverse axis is possible, so that a main body will then be obtained which is shaped and adapted to the anatomical circumstances, preferably having a curved design.

Depending on the condition of the bone to be stabilized, it may become necessary to employ wire, in addition to the angled profiles and their screw connections. In order to ensure the precise positioning of the wire, the invention provides that the recesses are designed so as to affix any possibly needed cerclage wire, or that a shaped article encompassing the cerclage wire and to be installed into the recesses is provided. This kind of use of the cerclage wire in the described affixing is advantageous in particular when the bone has lost stability across its length, for example, due to a corresponding illness (e.g. osteoporosis, tumor, periprosthetic fractures), so that it restricts, or does not admit the installation of drill holes.

An additional possibility for adapting the deformability of the angled profile to existing circumstances, is to design the recesses from the free edge of the main body so as to extend up to the remaining, sufficiently wide back, and preferably both plate legs are designed as with parts arranged offset to each other. This will provide the possibility to affect exclusively the back for the deformation, while the parts of the plate leg distant from it need not be involved in the deformation.

In order thereby to ensure the affixing of the angled profile to the bone, the invention provides that the tongue-like or lashing-like parts of the plate legs protruding from the back are rounded on one end and feature drilled affixing holes and/or are designed as varying in plate thickness with respect to the thickness of the back, and/or as having different lengths (even in groups). Due to the rounded design of the protruding parts, the potential for injury is reduced, and due to the different thickness of these protruding parts an additional, optimum adaptation to the circumstances at the bone is possible.

Similar to the prior art with regard to the flat, plate-shaped main bodies, the main bodies of the invention also provide that drilled holes are pre-fabricated and this provides the possibility to secure the main body to the bone with screws. In this regard the middle back of the angled profile is designed as flat and wide enough that corresponding holes can be well situated on the back. As a rule, the back is easiest to reach when tightening the screws, and indeed also without one having to perform too much preparatory surgery.

Regarding the designs of the main body described above, it is assumed that the particular plate legs are of equal length or also are designed to have an equal shape, and accordingly also the described recesses. But according to another variant, it is also possible that the plate legs have different lengths in correspondence to the bone to be connected to them. Thus an optimum adaptation to the anatomical circumstances of the particular bone is possible, and specifically both due to corresponding, supplemental shaping during the operation or also previously at the manufacturer.

Depending on the use of the main body it is possible to deviate slightly from the angular-shaped design of the main body and instead, to employ a semi-circular main body.

It has been pointed out above that the drill holes for the insertion or installation of the affixing screws are located on the middle comb or back. According to another embodiment of the invention, it is an advantage that in addition to the drilled holes on the back, across the length of the main body and distributed at a safety interval to the edges, affixing holes are provided which form preferably a static-angle structure. Thus it is possible, depending on the circumstances, to supply these affixing screws only on the back, or additionally or even only on the longitudinal flanks and/or plate legs, since there will be drill holes available at practically all important sites. Since the main body has an inherent stability, it may be quite sufficient to supply the drilled holes quasi only at the end regions or only in the middle, in order to screw the affixing screws into the bones.

It is also possible that the recesses and the intermediate webs formed between them will feature approximately the same width, whereas the edge webs are designed as twice as wide. In case of deformations during the surgery, this will have a particularly favorable effect, because thus a uniform deformation or flexure can be attained, without any significant loss of stability. The uniform design of the intermediate webs and of the recesses facilitates a uniform shaping, especially if flexures become necessary in the transverse axis of the main body.

Specifically with regard to the frequently mentioned required flexures in the longitudinal axis, or even in the transverse axis, it is an advantage that the drilled holes or even the affixing holes do not hinder the screwing in of affixing screws. This is attained in that the drilled holes and/or the affixing holes are designed so as to create a stable-angle receptacle for the screws. It makes no difference whether attachment screws are screwed into the holes drilled in the back, or into affixing holes, and whether precisely at the flexure midpoint, it is thus ensured that the attachment screws can also be securely and precisely positioned.

Depending on the formation of the bone, it may be practical to work with several short angled profiles, and this is possible in that the main body is designed so as to be recessed on one side to enable an overlapping with other main bodies, or it features specifically shaped connecting elements. This provides the possibility, for example, to work with standardized main bodies, which are then combined by joining to other main bodies, or to specially shaped connecting elements, in order to obtain an optimum overall main body.

In order also to include specially shaped bone regions in the stabilization, it is an advantage that the main body or the connecting elements feature retaining elements protruding at the ends, and at the end away from the plate are designed to accommodate a bolt or affixing screw and are adapted to the shape of the proximal ulna, of the distal or proximal femur, of the clavicula or other bone and are designed and arranged so as to protect tendons. Specifically with regard to the bones in the elbow region, with the protruding retaining elements the bones can be encompassed on the side in a favorable manner, wherein then the two retaining elements are connected via a bolt located behind the strong tendon so that the desired stability is obtained. Similarly, the use of a connecting element with the protruding retaining elements is advantageously possible for other bones as well.

A main body of this kind can obtain its base form by means of bending, edging, pressing or shaping, wherein it is manufactured practically from a surgical steel, titanium or a specially stabilized plastic.

It is an advantage that the main body is designed to be connected to other main bodies, to have a variable angle, to be equipped with parts shaped onto the side, and is designed with affixing holes formed as elongated holes along the longitudinal direction of the back. They can be either supplied with the appropriate shape, or can be brought into the required shape during the operation, as applies to the main body generally.

During the operation, the tendons, nerves or blood vessels running to the bone require particular consideration. Whereas in the prior art the known plates have to be inserted under the tendons, nerves or blood vessels, the invention provides that the main body is designed in advance, or can be shaped during surgery, so as to form a bridge over the tendons, nerves or blood vessels running to the bone, or that a main body designed as a bridge part with one or two main bodies can be arranged to bridge the tendons and can be connected to the main bodies. The operation itself is significantly simplified because it is complicated to push these comparatively large main bodies under a tendon, which itself is securely anchored to the bone, for example.

In particular the invention is characterized in that now for the first time an implement is provided for stabilization of bone fractures which guarantees stability even for complicated comminuted fractures, which is absolutely necessary for an early functional exercise of the neighboring joints. Due to the transition from plate-shaped main bodies to angular-shaped main bodies, it is possible to support the stabilization of the bone region so effectively that during subsequent working, but in particular during the exercise of the neighboring joints, adverse impacts cannot occur, in particular there will not be a renewed displacement of the bone fragments. An added advantage is that angled main bodies of this kind can be well attached to the bone region being stabilized, wherein due to the special configuration of the angled main body and the formation of required drill holes and recesses precisely at those sites where they will be needed later, the processing of these main bodies on the bone is possible in an optimum manner. Moreover, it is an additional advantage that the angled main body in the bone region being stabilized can be well adapted in its deformation about the longitudinal axis and also about the transverse axis, which is additionally facilitated due to the large recesses. Unevennesses are provided on the inside of the plate legs which ensure a point by point contact of the angled profile on the bone, so that the blood flow is mostly maintained. The described recesses improve the intraoperative adaptation of the angled profile to the anatomical circumstances. The adaptation and/or deformation of the angled profile is possible by means of specially constructed tongs to manipulate the plate holes or the recesses or the additionally provided attachment elements, even during an operation. Given an unfavorable structure or condition of the bone, e.g. due to an illness, osteoporosis, tumor or in the presence of prosthesis, cerclage wires can be used instead of or in addition to, which are affixed exactly in the right position by means of the special angled profiles. The mentioned formation of an angled profile with solid back and protruding, tongue-like parts allows a specific deformation in “all” directions. The tongue-like parts designed at an offset ensure a reliable screw connection, even when using all holes, since the screws cannot touch each other.

Additional details and advantages of the subject matter of the invention are evident from the following description of the associated drawings, which depict preferred exemplary embodiments with their relevant details and individual parts. The figures show:

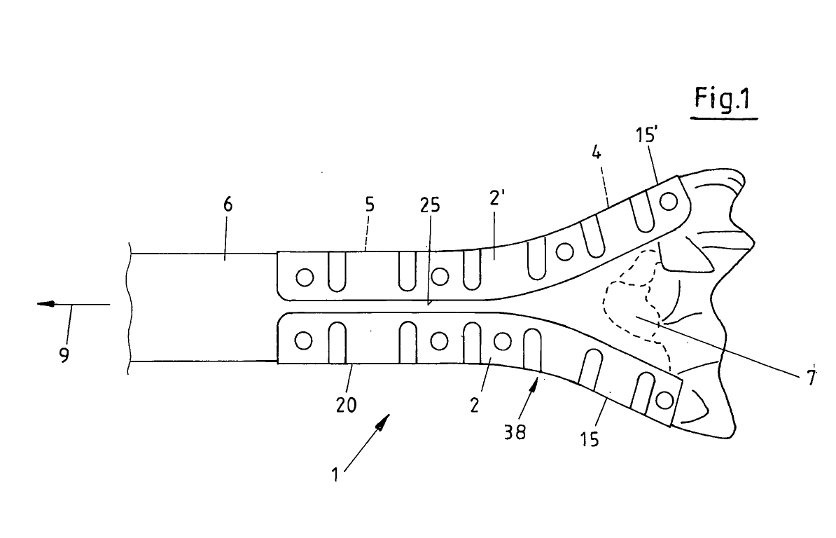

FIG. 1 a bone with a bone region to be stabilized, wherein affixing screws have not yet been installed, but the implements in the form of angled main bodies are already resting against the bone,

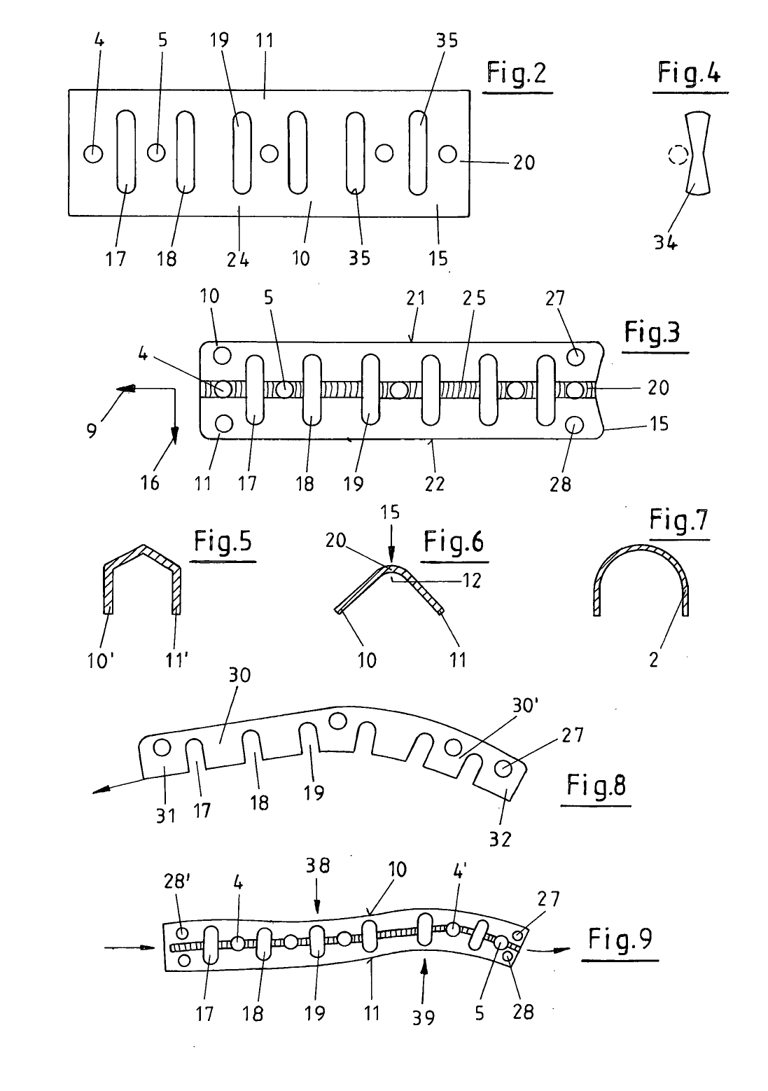

FIG. 2 a plate-shaped main body shown in top view,

FIG. 3 an angled main body with middle comb and/or back,

FIG. 4 an X-shaped recess,

FIG. 5 a multi-step angled main body,

FIG. 6 an angled main body including a 90°-angle,

FIG. 7 a curved main body,

FIG. 8 an anatomically correct main body curved or coiled around the longitudinal axis,

FIG. 9 a main body curved around the transverse axis at two places,

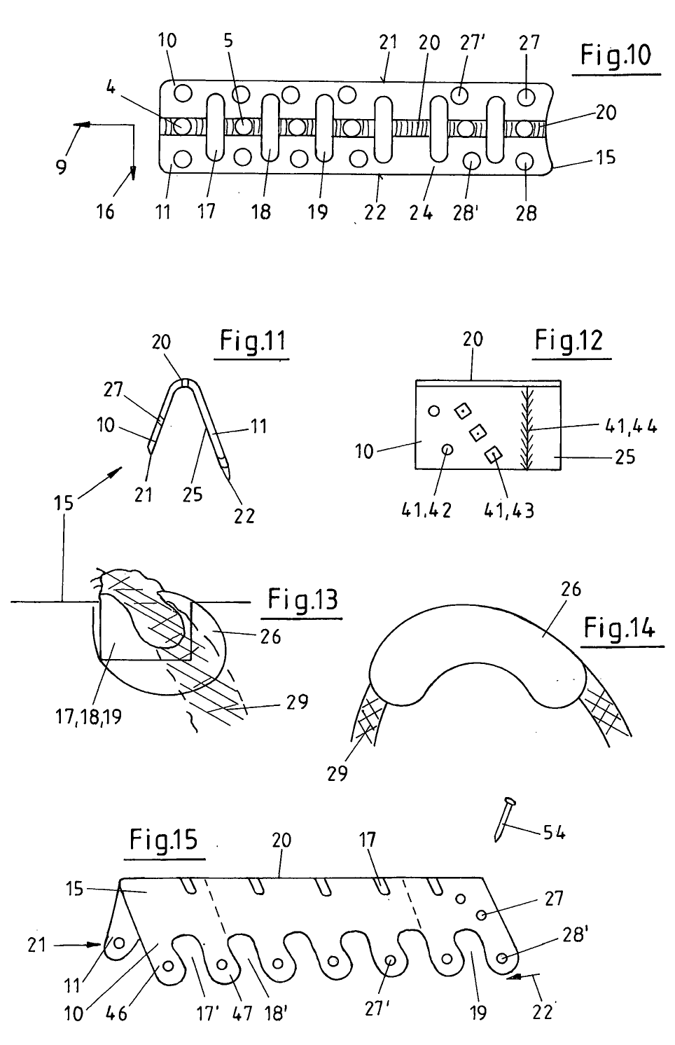

FIG. 10 the angled main body known from FIG. 3 with multiple drilled attachment holes,

FIG. 11 an angled profile with different length plate legs,

FIG. 12 a top view of the inside of a plate leg with unevennesses,

FIG. 13 a recess with inserted shaped part for a cerclage wire,

FIG. 14 a cross section of a recess according to FIG. 13,

FIG. 15 an angled profile with solid back and tongue-like parts of the plate leg rounded on the end and emanating from the back,

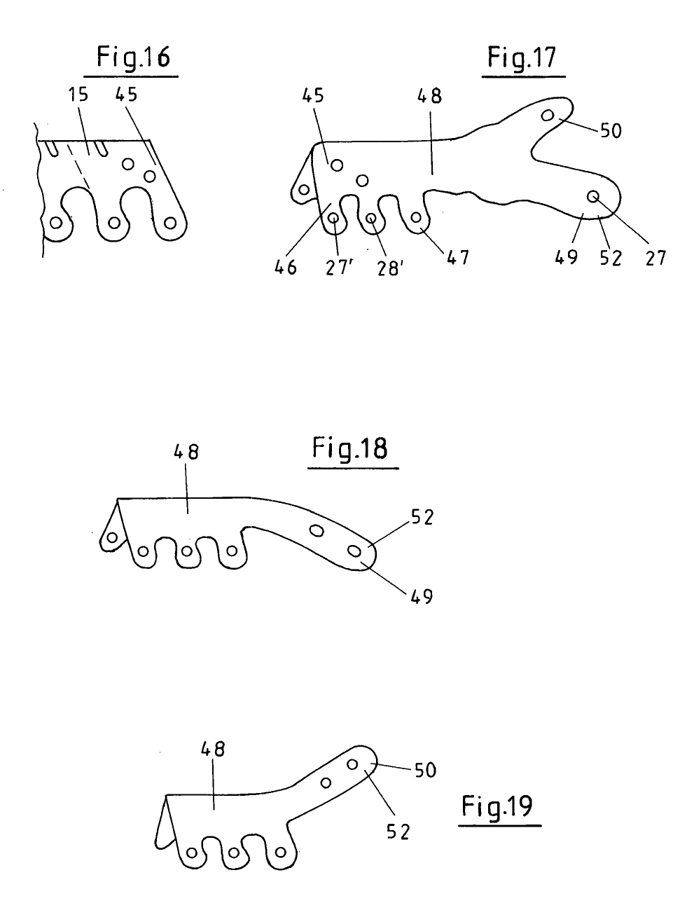

FIG. 16 an angled profile which can be pushed onto another angled profile and/or connecting element,

FIG. 17 a connecting element shown in perspective view,

FIG. 18 an additional connecting element,

FIG. 19 a connecting element with retaining element,

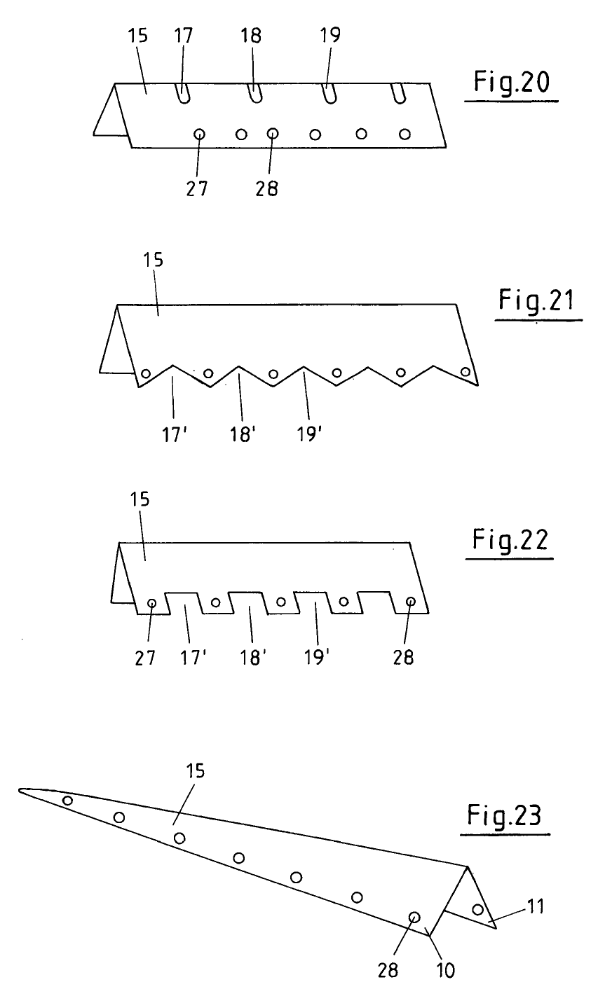

FIG. 20 an angled profile with recesses in the back region,

FIG. 21 an angled profile with recesses in the edge region of the plate leg, in angled format,

FIG. 22 an angled profile with rectangular recesses in the edge region,

FIG. 23 an angled profile designed to taper to a point, shown in perspective view,

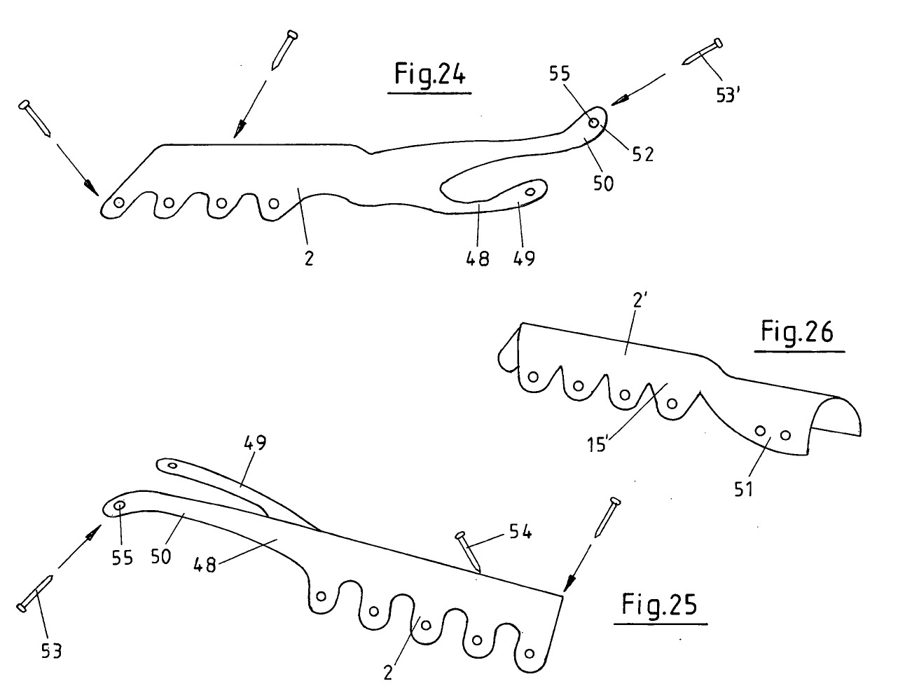

FIG. 24 an angled profile with arm-like connecting elements,

FIG. 25 an angled profile with especially long arm-like connecting elements,

FIG. 26 an angled profile with recess facilitating a connection to other angled profiles,

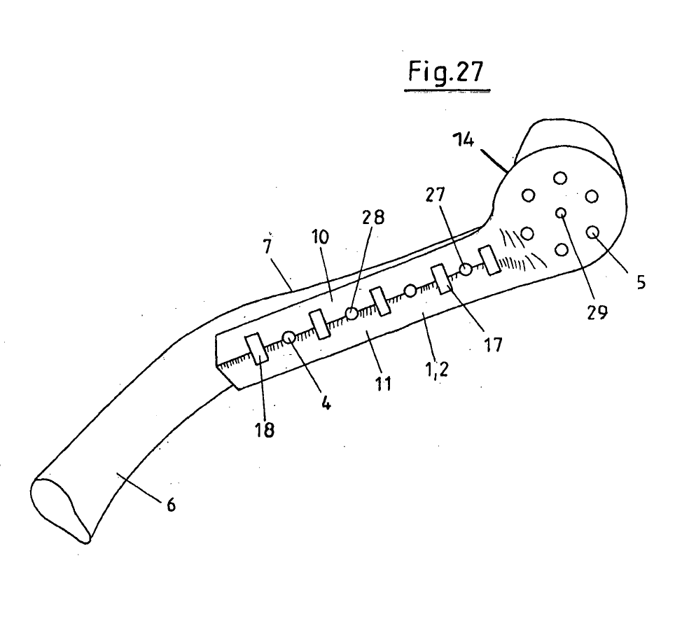

FIG. 27 an angled profile passing into a bulged round part,

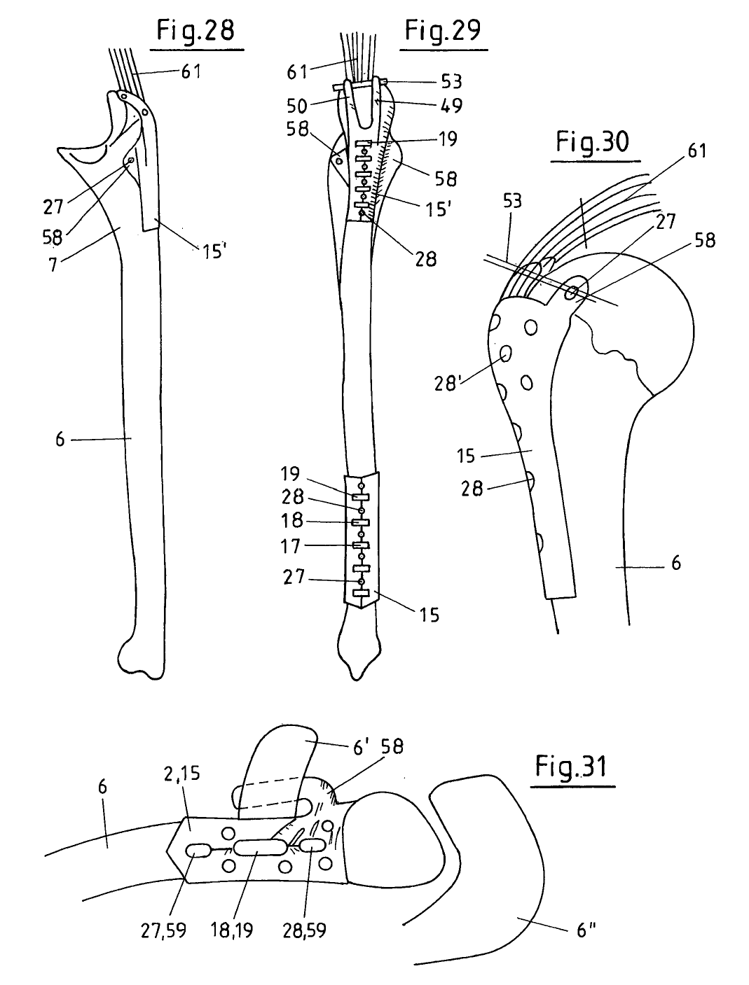

FIG. 28 an angled profile with a part molded onto the side, shown in side view,

FIG. 29 the corresponding angled profile shown in top view and a separate angled profile, without molded on parts, supplied under it,

FIG. 30 an angled profile with additional molded on parts for a bolt,

FIG. 31 an angled profile with a molded on part engaging with an additional bone from below,

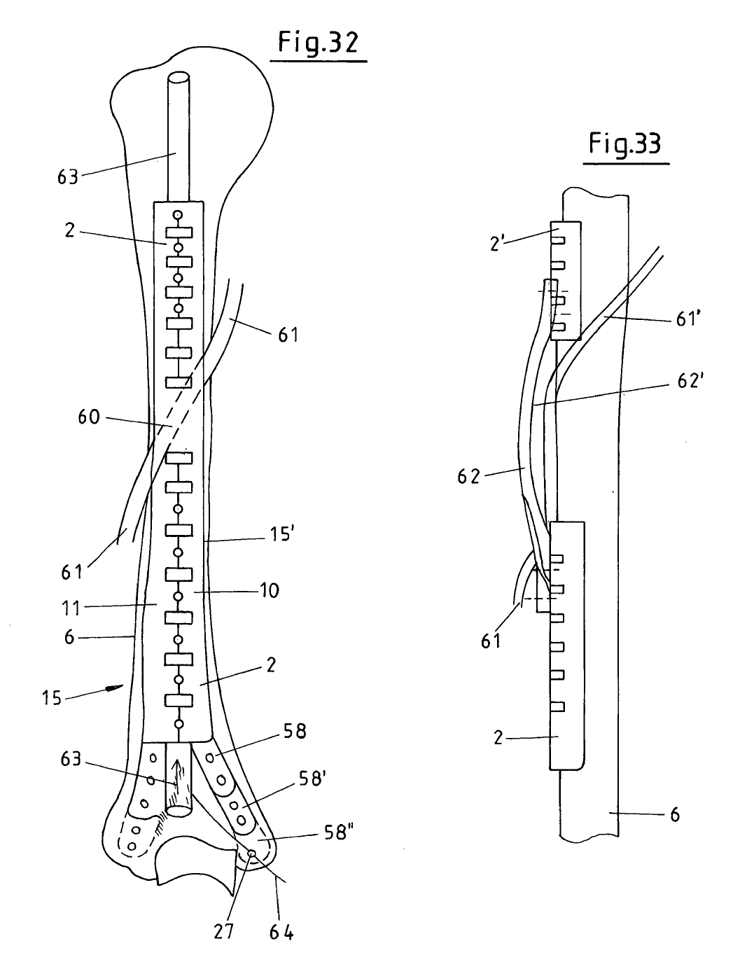

FIG. 32 an angled profile covering a tendon and

FIG. 33 a structure consisting of several angled profiles with additional bridge part.

FIG. 1 depicts a bone 6, here, an elbow joint which must be surgically stabilized due to a complicated comminuted fracture. A comminuted fracture of this kind, which is indicated here by dotted lines, may have occurred due to a traffic or workplace or even sports accident. The broken bone fragments have been displaced due to the accident and in the depiction according to FIG. 1 are to be replaced into their original position during an operation. Now in order to hold them in this position and to allow the possibility of an early functional exercise of the neighboring joint, without a renewed shifting of these broken bone fragments or broken bone pieces, implements 1 in the form of plate-shaped main bodies 2, 2′ are to be placed in the bone region 7 to be stabilized. These implements 1 pertain to main bodies 2, 2′ which are edged off or bent off uniformly in the longitudinal axis 9, in order thus to form a given angle corresponding to the bone region 7. The given angle is depicted in FIG. 6. It is also better seen in FIG. 6 that due to the bending or edging 5 at the given angle 12, two equal plate legs 10, 11 are produced, which feature drilled holes at several locations, which are not identified by reference numbers. Thus the main body 2 is formed as an angled profile 15 that has been adapted exactly to the shape of the bone region 7 being stabilized, by means of a bending about the bending site 38. The drilled holes 4, 5 formed at the back 20 are merely indicated here. This is preferably where the needed attachment screws are installed.

The angled main bodies 2, 2′ shown in FIG. 1 are formed from plate-shaped main bodies, wherein FIG. 2 shows that the drilled holes 4, 5 and also additional recesses 17-19 are incorporated into the main body 2 even before the bending or edging. This flat main body 2 is then edged according to FIGS. 3 and 6, so that an angled profile 15 is produced. The recesses 17-19 feature a flat-oval shape 35, wherein as illustrated with the aid of FIG. 4, instead of the flat-oval shape 35, an X-shape 34 can also be selected.

As already mentioned, FIG. 3 shows the edged design of the main body 2, wherein a central element known as a middle comb or back 20 is produced which has a width sufficient to install a number of drilled holes 4, 5 in order thus to facilitate the specific incorporation of attachment screws, and specifically where the drilled holes 4, 5 are usually most easily reached during installation of the screws. In FIG. 3 the longitudinal axis is denoted by reference number 9, and the transverse axis is identified by reference number 16. According to FIG. 6, the main body 2 is edged around the longitudinal axis 9 in the region of the back 20, whereas according to FIG. 5, a multiple edging is used, and according to FIG. 7, a round bending produces a kind of semi-circle. These various shapes are intended to correspond to the configuration of the bone 6 to be stabilized.

FIG. 3 and also FIG. 2 illustrate that the selected recesses 17-19 emanate from the back 20 and extend up to the edges 21, 22. They are continuous in the region of the back 20 of the plate legs 10, 11. Thus the bending around the longitudinal axis 9, but especially also around the transverse axis 16 is facilitated, without the stability of the plate legs 10, 11 and/or of the angled profile 16 being significantly affected thereby.

In addition to the already mentioned drilled holes 4, 5 in the region of the back 20, additional affixing holes 27, 28 are provided especially at the corners of the main body 2. Whether or not these holes will be needed in the operation will be determined for each individual case. But in any case the possibility is obtained to provide affixing screws where the bone 6 can be best reached in a particular case, and where the bone 6 also provides the needed possibility of properly accommodating the affixing screws, that is, where there is sufficient material.

FIG. 8 shows a main body 2, that is, an angled profile 15, which per se is curved around the longitudinal axis 9 in order to obtain the shape according to FIG. 1, for instance. The recesses 17-19 have a favorable effect here because they admit this bending process. Also illustrated on the basis of FIG. 8 is that the intermediate webs 30 between the individual recesses 17-19 are of about the same size, whereas the edge webs 31, 32 have a broadened design. The purpose of this is, firstly, to easily accommodate the described affixing holes 27, 28, and secondly, to assure the stability of the overall main body 2.

In addition to the deformation according to FIG. 8, a deformation about the transverse axis 16 is also possible (FIG. 3), as is additionally depicted in FIG. 9. In this case even two flexure sites 38, 39 are provided, in order to adapt exactly to the anatomy of the bone region 7 to be stabilized. The configuration according to FIG. 9 is also fostered in that the main body 2 is preset as an angled profile 15 and features the recesses 17-19.

FIG. 10 is constructed similar to FIG. 3, but here additional affixing holes 27′, 28′ are provided. These affixing holes 27′, 28′ can be arranged at a right angle to the edge 21, 22, or even running at a slant thereto. In the region of the edge 21, 22 between the recesses 17-19 there remains a spacing 24 in order thus to keep the stability of the entire main body 2 as great as possible. FIG. 11 illustrates that the two plate legs 10, 11 need not necessarily have the same length. Rather, the length is chosen according to the particular install site, wherein affixing holes 27 are additionally indicated here.

In order to have preferably a point by point contact of the main body 2 and/or of the plate legs 10, 11 with the periosteum, according to FIG. 12 the invention provides that unevennesses 41 are provided on the plate side 25, thus quasi on the inside of the plate legs 10, 11. Unevennesses 41 are depicted in FIG. 12 which appear in the shape of knobs 42, pointed or pyramid-shaped protrusions 43, or even as straight or slanting grooves 44.

Where additional cerclage wire 29 has to be used, for example, due to not entirely stable bone material, then this wire must be secured in place by a special configuration and/or formation of the angled profiles 15 by means of shaped parts 26 inserted into the recesses 17-19. In this way the cerclage wire 29 is affixed in a favorable manner and can be tightened so that it pulls the bone 6 together so that at the same time, the securing of the angled profile 15 is also achieved. FIG. 13 shows a side view and FIG. 14 a cross section of this particular design of the angled profile 15.

Whereas the region of the edges 21, 22 in the previously depicted angled profiles displays a consistent, rod-shaped part, according to FIG. 15 and FIGS. 16, 17, the edge region is slotted in order to facilitate the bending of the angled profile 15. According to FIG. 15, recesses 17 are also provided in the region of the back 20 neighboring the protruding parts 46, 47 of the plate legs 10, 11. The parts 46, 47 have a tongue-like formation and are rounded or angular in the end region and are additionally provided with affixing holes 27′, 28′. The recesses forming between the tongue-like parts 46, 47 are identified with reference numbers 17′, 18′, 19′.

In the end region of the angled profile 15 there are drilled affixing holes 27 which can also be designed so that a connection is possible to a second angled profile 15′ which is pushed in from above or below.

FIG. 16 shows the connecting region 45 of an angled profile 15 which is shaped to correspond to the connecting region 45′ of a neighboring connecting element 48. This connecting element 48 features at its opposite ends the retaining elements 49, 50, which allow an optimum connection to specially shaped regions of a bone 6, in particular the bone ends. Both the angled profile 15 and also the connecting element 48 feature the tongue-like parts 46, 47 on their edges, as mentioned above, which here too feature affixing holes 27′, 28′ on their ends. Likewise, the retaining elements 49, 50 arranged on the far end 52 of the connecting element 48 away from the plate, are equipped with these affixing holes 27.

FIGS. 18 and 19 show other design embodiments of the connecting elements 48, wherein each of the two embodiments features only one retaining element 49 or 50, respectively.

To illustrate that different designs of the angled profiles 15 are possible, especially in the edge region, FIGS. 20 to 23 depict angled profiles, wherein in one case affixing holes 27, 28 are provided in the edge region and in FIGS. 21 and 22 additional recesses 17′-19′ are provided. This produces the mentioned tongue-like parts 46, 47, or also the acute-angle or rectangle parts according to FIGS. 21 and 22. FIG. 23 is a special design in which the two smooth legs 10, 11 are designed to come together at one end.

FIG. 24 and FIG. 25 depict a special design of a connecting element 48, in which the retaining elements 49, 50 are of sufficiently long design, and for example can encompass the head of a bone 6, so that the bolt denoted as 53 or 53′, respectively, which connects the two retaining elements 49, 50, can be inserted behind the head without affecting the tendon (not depicted) located in front. The retaining elements 49, 50 feature drilled holes 55 at their ends 52 away from the plate, through which a bolt 53 can be inserted. Reference number 54 denotes an affixing screw which is to be screwed into one of the affixing holes 27, 28. Reference number 51 in FIG. 26 denotes a region of an angled profile 15 which forms a kind of lug in order to facilitate pushing on of the connecting element 48′ according to FIG. 24. In this way the length of the main body 2 and/or implement 1 can be easily and quickly adapted to the circumstances, without adversely impacting the stability of the overall structure. The advantage is that due to this design, a modular and simple construction of this kind of implement 1 is possible.

FIG. 27 shows a bone 6 whose angled profile 15 passes over into a bulged profile in the region of the bone end 14 and which is adapted to the shape of the bone end 14. In the middle of this bulged, round part there is a drilled hole through which a wire, for example a cerclage wire 29, can be passed before the other affixing holes 27, 28 are installed. The advantage is that after introduction of the cerclage wire 29, the remaining angled profile 15 can be rotated until the shaft of the bone 6 has been precisely grasped and it can now be secured with screws.

FIGS. 28 and 29 depict a bone which features at the lower end a simple angled profile 15 which has the same design on both ends, whereas the angled profile 15′ allocated to the upper end of the bone 6 is equipped with lateral molded parts 58 and terminal retaining elements 49, 50, in order thus to grasp this specially shaped bone and not to affect the tendon denoted by reference number 61.

FIG. 30 depicts an angled profile 15 which features at its upper end a bulged end part with the protruding molded parts 58 engaging behind the tendons, wherein it is particularly clear here that with the angled profiles 15 according to the invention, components can be produced with little expense which are or can be particularly well adapted to the bone under treatment.

In turn, FIG. 31 depicts an angled profile with specially provided recesses 18, 19 and also affixing holes, wherein here the recess 18 is shown in the shape of an especially long rectangle. A separate molded part 58 is assigned to this angled profile 15 and has an arm-like design and can grasp another bone 6′. The affixing holes 27, 28 allocated to the recess 18 in this case have the shape of an elongated hole 59 in order to facilitate a simpler adaptation to the affixing point.

With regard to the bone 6 depicted in FIGS. 32 and 33, a tendon 61′ runs close to the bone 6 and is covered here by a bridge structure which eliminates the need to shield the tendon 61′.

In FIGS. 32 and 33, a long nail 63 is introduced into the bone 6. In the depiction according to FIG. 32, the mentioned tendon 61′ is bridged by a one-part angled profile 15′, wherein this angled profile 15′ has a bridge-like design in the region of the tendon 61′. Recesses for the tendon 61′ are provided either in the lateral plate legs 10, 11, or the angled profile 15 is flattened in the bridge region. In the representation according to FIGS. 32 and 33, the tendon 61′ is depicted as larger, but of course there can also be a thinner or even a thicker tendon. In any case, it can be bridged with the construction in FIG. 32, and equally also with the construction shown in FIG. 33, wherein according to FIG. 33 it is shown that two individual main bodies 2, 2′ are spanned by the bridge part 62, wherein this bridge part 62 features drilled holes at least on the end, in order to allow a connection to the two main bodies 2, 2′. The bridge part 62 is smoothed on the underside in order to avoid injury to the tendon 61′.

At the lower end of the bone 6 in FIG. 32, different length and possibly differently shaped molded parts 58′, 58″ are provided on a main body 2 and/or an angled profile 15 corresponding to the formation of the bone 6. The molded part 58″ shown on the bottom of the figure here features an affixing hole 27 through which a slanting screw 64 is inserted; this is an advantage in particular when a nail 63 is used, as shown here.

All named features, including those indicated in the drawings alone, are deemed to be essential to the invention, either alone or in combination.

1. Implements for surgical treatment and in particular for stabilization of bone fractures, consisting of a plate-shaped main body (2), in which drilled holes (4, 5) are provided for fixation of the main body (2) to the bone (6) by means of screws, and which is manufactured from a material suitable for humans,
wherein
the length and width of the main body (2) is designed to correspond to the bone region (7) being reinforced therewith and to have a resulting angular profile (15), wherein the two plate legs (10, 11) thereof are shaped to encompass an angle (12) adapted to the associated bone region (7).

2. Implements according to claim 1,
wherein
the main body (2) features elongated recesses (17-19) which are designed to extend from the back (20) formed by the two plate legs (10, 11) up to the edges (21, 22) of the plate legs (10, 11) and to leave a protective separation (24).

3. Implements according to claim 2,
wherein
the recesses (17-19) feature a width and spacing to each other that facilitates the edging or bending of the main body (2) about the longitudinal axis (9) and/or a deformation of the main body transverse to the longitudinal axis.

4. Implements according to claim 1,
wherein
the plate legs (10, 11) feature unevennesses (41) distributed along the plate side (25) resting against the bone (6).

5. Implements according to claim 4,
wherein
the unevennesses (41) are designed as nipple-like knobs (42) or also as conical or pyramid-like or cube-like protrusions (43) or even as grooves (44).

6. Implements according to claim 5,
wherein
the angled profile (15) is designed with an included angle of 90°.

7. Implements according to claim 6,
wherein
the angled profile (15) features an angle (12) greater than or less than 90°, adapted to the anatomical install site, wherein the angle (12) can also vary at individual locations of the angled profile (15).

8. Implements according to claim 1,
wherein
the recesses (17-19) are distributed regularly along the back (20) of the angled profile (15) or are concentrated at defined sites and are not provided at all at other sites.

9. Implements according to claim 3,
wherein
the main body (2) is shaped so as to be adapted to the anatomical circumstances, preferably as a curved design.

10. Implements according to claim 1,
wherein
the recesses (17-19) are designed so as to affix any possibly needed cerclage wire (29), or that a shaped article (26) encompassing the cerclage wire (29) and to be installed into the recesses (17-19) is provided.

11. Implements according to claim 1,
wherein
the recesses (17′, 18′, 19′) are designed so as to extend up to the remaining sufficiently wide back (20) and are designed preferably offset to each other in both plate legs (10, 11).

12. Implements according to claim 11,
wherein
the tongue-like or lashing-like parts (46, 47) of the plate legs (10, 11) protruding from the back are rounded on one end and feature drilled affixing holes (27′, 28′) and/or are designed as varying in plate thickness with respect to the thickness of the back, and/or as having different lengths (even in groups).

13. Implements according to claim 1,
wherein
the plate legs (10, 11) have different lengths in correspondence to the bone (6) to be connected therewith.

14. Implements according to claim 1,
wherein
in addition to the drilled holes (4, 5) on the back (20), across the length of the main body (2) and distributed at a safety interval to the edges (21, 22), affixing holes (27, 28) are provided which form preferably a static-angle structure.

15. Implements according to claim 1,
wherein
the main body (2) is designed so as to be recessed on one side to enable an overlapping with other main bodies (2′), or features specifically shaped connecting elements (48).

16. Implements according to claim 15,
wherein
the main body (2) or the connecting elements (48) feature retaining elements (49, 50) protruding at the ends, and at the end (52) away from the plate are designed to accommodate a bolt (53) or affixing screw (54) and are adapted to the shape of the proximal ulna, of the distal or proximal femur, of the clavicula or other bones and are designed and arranged so as to protect tendons.

17. Implements according to claim 1,
wherein
the main body (2) is manufactured from a surgical steel, titanium or a specially stabilized plastic.

18. Implements according to claim 1,
wherein
the main body (2) is designed to be connected to other main bodies (2′), to have a variable angle (12), to be equipped with parts (58) shaped onto the side, and is designed with affixing holes (27, 28) formed as elongated holes (59) along the longitudinal direction of the back (20).

19. Implements according to claim 1,
wherein
the main bodies (2) can be shaped so as to bridge tendons (61), nerves or blood vessels running to the bone (6), or specifically are designed in advance to form a bridge (60), or that one of them is designed as a bridge part (62) with one or two main bodies (2, 2′) arranged to bridge over the tendon (61) and being connectable to the main bodies (2, 2′).
